SEQUENCE LISTING (1 sequence)

SEQ ID NO: 1 - protein, 174 aa, Homo sapiens
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

(19) United States
(12) Patent Application Publication
(10) Pub. No.: US 2006/0045867 A1
(43) Pub. Date: Mar. 2, 2006

(54) Regeneration method for treating heart diseases and vascular system diseases using medicament

(76) Inventors: Hisayoshi Fujiwara, Gifu-shi (JP); Yu Misao, Gifu-shi (JP)

Correspondence Address: NIXON & VANDERHYE, PC, 901 NORTH GLEBE ROAD, 11TH FLOOR, ARLINGTON, VA, 22203, US

(21) Appl. No.: 10/924,197

(22) Filed: Aug. 24, 2004

(51) Int. Cl. A61K 38/19

(52) U.S. Cl. 424/85.1

(57) ABSTRACT

A regeneration method using a medicament against heart diseases and vascular system diseases, which comprises administering a polypeptide having granulocyte colony-stimulating factor activity under bone marrow suppressed conditions.

### BACKGROUND OF THE INVENTION

1. Field of the Invention

The present invention relates to a regeneration method for treating heart diseases and vascular system diseases by using a medicament.

2. Brief Description of the Background Art

As the regeneration methods for treating heart diseases and vascular system diseases so far reported, therapeutic methods are known in which adult stem cells mainly existing in the marrow are collected and separated and then injected into heart and vein tissues directly or through a coronary artery.

Granulocyte colony-stimulating factor (hereinafter also referred to as “G-CSF”) is a polypeptide which proliferates or differentiate neutrophilic granulocyte precursor cells and activates mature neutrophils. G-CSF is mainly used for the acceleration of increase of neutrophils in bone marrow transplantation, neutropenia caused by chemotherapy of cancer, osteomyelodysplasia syndrome, aplastic anemia, congenital or idiopathic neutropenia, human immunodeficiency virus (HIV) infection and the like (Shorei ni Manabu G-CSF no Rinsho (Clinic of G-CSF Learned from Cases), edited by Yasuo Ikeda, published by Iyaku Journal, Osaka, pp. 64-92 (1999)).

In recent years, it has been reported that when G-CSF is administered to a rabbit which caused myocardial infarction by ischemia reperfusion, bone marrow-originated stem cells are mobilized in peripheral blood, and regeneration of heart muscle and angiogenesis are generated while simultaneously accelerating the healing process to thereby improve the cardiac function [Circulation, 106 (supple. II), II-132-II-133 (2002)]. However, regeneration of heart and vein tissues by the single administration of G-CSF is not sufficient.

It has been reported that a stem cell capable of differentiating into myocardial cells or vascular endothelial cells is present in the mouse bone marrow, in addition to the hematopoietic stem cell which differentiates into hematopoietic tissues such as leukocytes and erythrocytes, and the mesenchymal stem cell which differentiates into mesodermal tissues such as skeletal muscle, fat cells and bone (WO01/48151), and it has been reported that mobilization of activated hematopoietic stem cells from bone marrow into peripheral blood is accelerated in mice to which cyclophosphamide and G-CSF have been administered [Blood, 97, 2278-2285 (2001)].

### SUMMARY OF THE INVENTION

An object of the present invention is to provide an effective regeneration method for treating heart diseases and vascular system diseases by using a medicament.

The present invention relates to the following (1) to (17).
(1) A method for treating heart diseases and vascular system diseases, which comprises administering a polypeptide having granulocyte colony-stimulating factor activity under bone marrow suppressed conditions.
(2) The method according to (1), wherein the polypeptide is a polypeptide comprising the amino acid sequence represented by SEQ ID NO:1.
(3) The method according to (1), wherein the polypeptide is a polypeptide which consists of an amino acid sequence in which at least one amino acid in the amino acid sequence represented by SEQ ID NO:1 is deleted, substituted and/or added, and has granulocyte colony-stimulating factor activity.
(4) The method according to (1), wherein the polypeptide is a polypeptide which consists of an amino acid sequence having a homology of 80% or more with the amino acid sequence represented by SEQ ID NO:1, and has granulocyte colony-stimulating factor activity.
(5) The method according to (1), wherein the polypeptide is a chemically modified polypeptide having granulocyte colony-stimulating factor activity.
(6) The method according to (5), wherein the modified polypeptide is a chemically modified polypeptide with polyalkylene glycol.
(7) The method according to (1), wherein the bone marrow suppressed conditions are bone marrow suppressed conditions which are caused by administration of an antitumor agent having bone marrow suppressive activity.
(8) The method according to (7), wherein the antitumor agent is at least one antitumor agent selected from the group consisting of an alkylating agent, a metabolic antagonist, an antitumor antibiotic, a plant pharmaceutical preparation and a platinum compound.
(9) The method according to (7), wherein the administration of the antitumor agent is administration of an alkylating agent and a metabolic antagonist in combination.
(10) The method according to (8), wherein the alkylating agent is cyclophosphamide.
(11) The method according to (9), wherein the alkylating agent is cyclophosphamide.
(12) The method according to (8), wherein the metabolic antagonist is 5-FU.
(13) The method according to (9), wherein the metabolic antagonist is 5-FU.
(14) The method according to (7), wherein the bone marrow suppressed conditions are bone marrow suppressed conditions in which count of leukocytes in peripheral blood is 90% or less of the count before the administration of the antitumor agent.
(15) The method according to (7), wherein the bone marrow suppressed conditions are bone marrow suppressed conditions in which count of leukocytes in peripheral blood is 75% or less of the count before the administration of the antitumor agent.
(16) The method according to (7), wherein the bone marrow suppressed conditions are bone marrow suppressed conditions in which count of leukocytes in peripheral blood is 60% or less of the count before the administration of the antitumor agent.
(17) The method according to (1), wherein the heart diseases and vascular system diseases are at least one of diseases selected from the group consisting of myocardial infarction, angina pectoris, heart failure, cardiac myopathy and obstructive arteriosclerosis.

### DETAILED DESCRIPTION OF THE INVENTION

In the present invention, the heart diseases and vascular system diseases include myocardial infarction, angina pectoris, heart failure, cardiac myopathy, obstructive arteriosclerosis and the like.

The polypeptide having G-CSF activity includes a polypeptide comprising the amino acid sequence represented by SEQ ID NO:1, or a polypeptide which consists of an amino acid sequence in which at least one amino acid residue in the amino acid sequence represented by SEQ ID NO:1 is deleted, substituted and/or added, and has G-CSF activity.

Examples include nartograstim (trade name: Neu-up, manufactured by Kyowa Hakko Kogyo Co., Ltd.), filgrastim (trade name: Gran, manufactured by SANKYO; trade name: Granulokine, manufactured by Hoffmann-La Roche; trade name: Neupogen, manufactured by Amgen), lenograstim (trade name: Neutrogin, manufactured by Chugai Pharmaceutical; trade name: Granocyte, manufactured by Aventis), pegfilgrastim (trade name: Neulasta, manufactured by Amgen), sargramostim (trade name: Leukine, manufactured by Schering) and the like.

Also, the polypeptide having G-CSF activity includes a polypeptide having a homology of preferably 60% or more, more preferably 80% or more, still more preferably 90% or more, and most preferably 95% or more, when its amino acid sequence homology with G-CSF having the amino acid sequence represented by SEQ ID NO:1 is searched by BLAST (Basic Local Alignment Search Tool). Examples of the polypeptides in which at least one amino acid residue in the amino acid sequence represented by SEQ ID NO:1 is substituted, which has G-CSF activity are shown in Table 1.

TABLE 1

| Position from the N- | | | | | | | | | | | | |
|---|---|---|---|---|---|---|---|---|---|---|---|---|
| terminus amino acid | Substituted amino acids in various proteins | | | | | | | | | | | |
| (G-CSF of SEQ ID NO: 1) | a) | b) | c) | d) | e) | f) | g) | h) | i) | j) | k) | l) |
| 1st (Thr) | * | Val | Cys | Tyr | Arg | * | Asn | Ile | Ser | * | Ala | * |
| 3rd (Leu) | Glu | Ile | Ile | Ile | Thr | Thr | Glu | Thr | Thr | * | Thr | * |
| 4th (Gly) | Lys | Arg | Arg | Arg | Arg | Arg | Arg | Arg | Arg | Arg | Tyr | * |
| 5th (Pro) | Ser | Ser | Ser | Ser | Ser | Ser | Ser | Ser | Ser | * | Arg | * |
| 17th (Cys) | Ser | Ser | Ser | Ser | Ser | Ser | Ser | Ser | Ser | Ser | Ser | Ser |

* unsubstituted amino acid

Furthermore, the polypeptide having G-CSF activity can be chemically modified.

The chemical modification method includes the method described in WO00/51626 and the like, and a polypeptide having G-CSF activity modified with, for example, polyalkylene glycol such as polyethylene glycol (PEG) is included.

In order to obtain bone marrow suppressed conditions before the administration of a polypeptide having G-CSF activity, an antitumor agent having bone marrow suppressive activity is preferably administered.

The term “an antitumor agent having bone marrow suppressive activity” means that the antitumor agent has influence upon bone marrow and activity of decreasing its function to produce blood (leukocyte, erythrocyte, platelet, etc.).

The antitumor agent having bone marrow suppressive activity includes an alkylating agent, a metabolic antagonist, an antitumor antibiotic, a plant pharmaceutical preparation, a platinum compound and the like.

The alkylating agent includes ifosfamide, cyclophosphamide, dacarbazine, nitrogen mustard N-oxide hydrochloride, nimustine hydrochloride, busulfan, melphalan, ranimustine and the like, and cyclophosphamide is preferred in the present invention.

The metabolic antagonist includes enocitabine, carmofur, cytarabine, cytarabine ocfosfate, tegafur, UFT, doxifluridine, 5-fluorouracil, hydroxycarbamide, procarbazine hydrochloride, methotrexate, mercaptopurine, gemcitabine hydrochloride and the like, and 5-fluorouracil is preferred in the present invention.

The antitumor antibiotic includes idarubicin hydrochloride, epirubicin hydrochloride, zinostatin stimalamer, daunorubicin hydrochloride, doxorubicin hydrochloride, pirarubicin hydrochloride, pepleomycin hydrochloride, bleomycin hydrochloride, mitomycin C and the like.

The plant pharmaceutical preparation includes irinotecan hydrochloride, etoposide, docetaxel hydrate, vincristine sulfate, pacritakicel, vinorelbine tartarate and the like, and pacritakicel is preferred in the present invention.

The platinum compound includes carboplatin, cisplatin, nedaplatin and the like, and cisplatin is preferred in the present invention.

Each component of the polypeptide having G-CSF activity or the antitumor agent having bone marrow suppressive activity used in the present invention can be administered alone, but generally, it is preferred to administer it to an animal (including human) as a pharmaceutical preparation produced by any method known in the technical field of manufacturing pharmacy, by mixing it together with at least one pharmaceutically acceptable carrier.

It is preferred to use a route of administration which is most effective in the treatment, and examples include oral administration and parenteral administration such as buccal, airway, rectal, intramuscular, subcutaneous, intradermal and intravenous administrations. Among these, intramuscular, subcutaneous, intradermal, intravenous or airway administration is preferred.

The dosage forms include tablets, capsules, granules, injections, ointments, tapes, dry powders, inhalations such as aerosols, and the like.

In the production of solid preparations such as tablets, for example, excipients such as lactose; disintegrating agents such as starch; lubricants such as magnesium stearate; binders such as hydroxypropylcellulose; surfactants such as fatty acid ester; plasticizers such as glycerol; and the like can be used.

The pharmaceutical preparation suitable for intramuscular, subcutaneous, intradermal, intravenous or airway administration includes injections, sprays and the like.

In the production of injections, for example, water, saline, plant oil such as soybean oil, solvents, solubilizing agents, tonicity agents, preservatives, antioxidants, and the like can be used.

Also, inhalations are prepared by using the polypeptide having G-CSF activity or the antitumor agent having bone marrow suppressive activity alone or together with a carrier or the like which does not irritate buccal and airway mucous membranes of the recipient and can facilitate absorption of the peptide or the antitumor agent by dispersing it as fine particles. The carrier includes lactose, glycerol and the like. Depending on the properties of the polypeptide having G-CSF activity or the antitumor agent having bone marrow suppressive activity and the carrier to be used, preparations such as aerosols and dry powders can be produced. Furthermore, the components exemplified as the additives of oral preparations can also be added to these parenteral preparations.

Although the dose and administration frequency of the polypeptide having G-CSF activity vary depending on the intended therapeutic effect, administration method, treating period, age, body weight and the like, generally, it is preferred to administer in a dose of 0.01 μg/kg to 10 mg/kg, preferably 1 to 100 μg/kg, and more preferably 1 to 10 μg/kg, per day per adult.

Also, the dose and administration frequency of the antitumor agent having bone marrow suppressive activity are appropriately determined so as to give bone marrow suppressed conditions at the time when the polypeptide having G-CSF activity is administered. The bone marrow suppressed conditions at the time when the polypeptide having G-CSF activity is administered include bone marrow suppressed conditions in which count of leukocytes in peripheral blood is 90% or less, preferably 75% or less, and more preferably 60% or less, of the count before the administration of the antitumor agent having bone marrow suppressive activity.

In the present invention, the antitumor agent having bone marrow suppressive activity can be used alone or as a combination of two or more.

Furthermore, in the present invention, when the antitumor agents having bone marrow suppressive activity are used as a combination of two or more, they can be used or administered as a single preparation (mixture preparation) or as a combination of plural pharmaceutical preparations, so long as the preparations are formulated so as to include the respective effective ingredients. When they are used as a combination of plural pharmaceutical preparations, they can be used or administered simultaneously or separately by keeping a period. Also, these pharmaceutical preparations can be used in the form of, for example, tablets, capsules, granules, injections, ointments, tapes, inhalations such as dry powders and aerosols, and the like.

When they are administered as a combination of plural pharmaceutical preparations, for example, (a) a component comprising the first antitumor agent having bone marrow suppressive activity and (b) a component comprising the second component having bone marrow suppressive activity can be separately formulated as described above to prepare a kit, and the respective components can be administered by using the kit simultaneously or separately by keeping a period, to the same subject through the same route or different routes.

The kit which can be used is a kit which comprises two or more containers (e.g., vial, bag, etc.) and the contents, wherein materials, shapes and the like of the containers are not particularly limited so long as they are such containers that denaturation of components as the contents by external temperature and light or dissolution of chemical components from the containers do not change during storage, and which also has such a mode that the above first component and second component as the contents can be administered through separate routes (e.g., tube, etc.) or the same route. Examples include kits of tablets, injections, inhalations and the like.

The above pharmaceutical preparations can be produced according to the conventional method by using pharmaceutically acceptable diluents, excipients, disintegrating agents, lubricants, binders, surfactants, water, saline, plant oil solubilizing agents, tonicity agents, preservatives, antioxidants and the like, in addition to the respective effective ingredients.

In the production of tablets, for example, excipients such as lactose; disintegrating agents such as starch; lubricants such as magnesium stearate; binders such as hydroxypropylcellulose; surfactants such as fatty acid ester; plasticizers such as glycerol; and the like can be used.

In the production of injections, for example, water, saline, plant oil such as soybean oil, solvents, solubilizing agents, tonicity agents, preservatives, antioxidants, and the like can be used.

Also, inhalations are prepared by using the antitumor agent having bone marrow suppressive activity alone or together with a carrier or the like which does not irritate buccal and airway mucous membranes of the receptor and can facilitate absorption of the antitumor agent it as fine particles. The carrier includes lactose, glycerol and the like. Furthermore, the components exemplified as the additives of oral preparations can also be added to these parenteral preparations.

As described above, the dose and administration frequency are appropriately determined so as to give bone marrow suppressed conditions at the time when the polypeptide having G-CSF activity is administered.

The validity of the method for treating heart diseases and vascular system diseases can be confirmed, for example, by the following method.

That is, a polypeptide having G-CSF activity is administered to an experimental animal such as a mouse, a rat, a rabbit or a monkey under bone marrow suppressed conditions, and its efficacy for heart diseases and vascular system diseases is confirmed by identifying alleviation of tissue injuries or accompanied symptoms and newly generated cells in the heart and vascular system tissues. The experimental animal is preferably an animal in which its heart and vascular system tissues are injured by a method such as ischemia reperfusion.

The newly generated cells in the heart and vascular system tissues can be detected, for example, by the following method.

A rabbit in which bone marrow-derived mononucleated cells labeled with a red fluorescence dye 1,1′-dioctadecyl-1 to 3,3,3′,3′,-tetramethylindocarbocyanine perchlorate (hereinafter referred to as “DiI”) have been returned to the bone marrow is prepared in advance, the heart and vascular system tissues of the rabbit are injured by a method such as ischemia reperfusion, bone marrow suppressed conditions are caused by administering an antitumor agent having bone marrow suppressive activity, and then a polypeptide having G-CSF activity is administered. The newly generated cells in the heart and vascular system tissues can be identified by measuring Di1 in the heart and vascular system tissues.

In addition, a bone marrow chimeric mouse in which, after irradiating a lethal dose of radioactive rays in advance, a bone marrow derived from a transgenic mouse of the same line capable of constitutively expressing green fluorescent protein (hereinafter referred to as “GFP”) was transplanted is prepared, the heart and vascular system tissues of the bone marrow chimeric mouse are injured by the above-described method, bone marrow suppressed conditions are caused by administering an antitumor agent having an bone marrow suppressive activity, and then a polypeptide having G-CSF activity is administered. The newly generated cells in the heart and vascular system tissues can be identified by measuring the fluorescence of GFP in the heart and vascular system tissues.

Examples of the present invention are shown below, but the present invention is not limited to these Examples.
1. Japanese white rabbits of about 2 kg in body weight (purchased from Chubu Kagaku Shizai) were used in the test, and each test group was organized by 30 rabbits. No clinically evident infection was observed in the rabbits. The test results were shown by the average value±the standard deviation, the significance test was carried out by ANOVA (Tukey-Kramer's test), and when the significance level was less than 5%, it was considered that there was a significant difference.
2. About 1.0×10⁸ of mononucleated cells were isolated and purified by using Ficoll (manufactured by JIMRO) from about 10 ml of the bone marrow fluid collected from the ilea of the rabbit, labeled with DiI (manufactured by Molecular Probes) by using dimethyl sulfoxide (DMSO, manufactured by Wako Pure Chemical Industries), and then returned to the iliac cavity. Five days thereafter, an ischemic state was generated for 30 minutes by ligating thick branches of the left side coronary artery, and then myocardial infarction was induced by reperfusion. On the 1st and 2nd days after the reperfusion, saline was intravenously administered to the rabbits of the 1st group and 2nd group, and 5-FU (15 mg/kg on the 1st day, 5 mg/kg on the 2nd day) and cyclophosphamide (40 mg/kg on the 1st day, 20 mg/kg on the 2nd day) to the rabbits of the 3rd group.
3. When the count of leukocytes in peripheral blood was measured on the 1st day of the reperfusion and on the 3rd day after the reperfusion, the count of leukocytes on the 1st day of the reperfusion was 9,456±1,206 cells/μl, while the count of leukocytes on the 3rd day after the reperfusion was 5,463±1,260 cells/μl. Based on these results, it was found that about 57.8% of the count of the leukocytes were bone marrow suppressed conditions in the 3rd group by the administration of 5-FU and cyclophosphamide. In this case, changes in the count of leukocytes on the 1st day of reperfusion and on the 3rd day after the reperfusion were not found in the 1st group and 2nd group.
4. During a period of from the 3rd day to the 7th day after the reperfusion, saline was subcutaneously administered to the rabbits of the 1st group once a day, and 10 μg/kg per animal of lenograstim (trade name Neutrogin, manufactured by Chugai Pharmaceutical) to the rabbits of the 2nd group and 3rd group.

On the 7th day after the reperfusion, both of the counts of mononucleated cells in peripheral blood of the 2nd group and 3rd group were about 1.5-fold larger than that of the 1st group. However, the count of mononucleated cells on the 14th day and 21st day after the reperfusion in the 2nd group was similar to that of the 1st group, while that of the 3rd group was about 2-fold larger than that of the 1st group.

As a result of flow cytometry, the counts of CD34-positive mononucleated cells in the peripheral blood of the 2nd group and 3rd group on the 7th day after the reperfusion were about 2.5-fold and 5-fold larger, respectively, than that of the 1st group, and the mononucleated cells were significantly increased in the 3rd group. On the 14th day and 21st day after the reperfusion, the increase disappeared in the 2nd group, but still maintained a high value in the 3rd group.

Based on the above results, it was found that CD34 -positive mononucleated cells capable of becoming precursor cells of the heart and vascular system tissues are increased in peripheral blood by the administration of a polypeptide having G-CSF activity under bone marrow suppressed conditions.
5. When ejection fraction of the left ventricle was measured by echocardiography on the 28th day after the reperfusion was 50±5% in the 1st group and 66.7±3.9% in the 2nd group, while it was 75.6±2.7% in the 3rd group, that is, both of the 2nd group and 3rd group showed significantly high values in comparison with the 1st group, and significant improvement was found in the 3rd group in comparison with the 2nd group.

Based on these results, it was found that cardiac function after myocardial infarction is markedly improved by the administration of a polypeptide having G-CSF activity under bone marrow suppressed conditions.
6. On the 28th day after the reperfusion, a rabbit was sacrificed to extract the heart, and then frozen sections were prepared from a boundary part between the infarction part and remaining heart muscle parts in the left ventricle risk region, and immunostaining was carried out by using mouse anti-troponin I monoclonal antibody (manufactured by Chemicon) which was an antibody which specifically reacts with myocardial cells. The ratio of the Di1-labeled myocardial cells to the troponin I-positive myocardial cells which was calculated by observing the immuno tissue specimens under a confocal laser microscope, it was 1.1±1.3% in the 2nd group, while the 3rd group showed a significantly high value of 8.9±9.1%.

Based on these results, it was found that regeneration of heart muscle after tissue destruction is markedly accelerated by the administration of a polypepti de having G-CSF activity under bone marrow suppressed conditions.

In addition, the frozen sections were subjected to immunostaining by using a monoclonal mouse anti-human endothelial cell CD31 antibody (manufactured by Dako) which was an antibody which specifically reacts with vascular endothelial cells and observed with a microscope under a 400× objective. As a result, the count of CD31-positive vascular endothelial cells per one visual field in the remaining heart muscle tissue contacting to the infarction part was 60±5 cells in the 1st group, 64±7 cells in the 2nd group and 120±8 cells in the 3rd group, and it was shown that the 3rd group had a significantly high value.

Based on these results, it was found that angiogenesis after the myocardial infarction is markedly accelerated by the administration of a polypeptide having G-CSF activity under bone marrow suppressed conditions.
7. After the heart tissue extracted on the 28th day after the reperfusion was subjected to formalin fixation and paraffin embedding, Masson trichrome staining was carried out and the fibrosis part which was as an old infarction region was quantitatively measured by using an image analyzer. As a result, the ratio of the infarction region occupying the left ventricle region was 20±9.2% in the 1st group, 13.5±8.1% in the 2nd group and 6.6±4.9% in the 3rd group. That is, the ratio was significantly low in the 2nd group in comparison with the 1st group, and was further decreased in the 3rd group and significant even in comparison with the 2nd group.

Based on these results, it was found that fibrosis after myocardial infarction is markedly suppressed by the administration of a polypeptide having G-CSF activity under bone marrow suppressed conditions.

As described above, it was found that administration of a polypeptide having G-CSF activity under bone marrow suppressed conditions markedly improves cardiac functions by further more accelerating regeneration of myocardial cells and blood vessels and suprression of fibrosis.

While the invention has been described in detail and with reference to specific embodiments thereof, it will be apparent to one of skill in the art that various changes and modifications can be made therein without departing from the spirit and scope thereof. All references cited herein are incorporated in their entirety.

What is claimed is:

1. A method for treating heart diseases and vascular system diseases, which comprises administering a polypeptide having granulocyte colony-stimulating factor activity under bone marrow suppressed conditions.

2. The method according to claim 1, wherein the polypeptide is a polypeptide comprising the amino acid sequence represented by SEQ ID NO:1.

3. The method according to claim 1, wherein the polypeptide is a polypeptide which consists of an amino acid sequence in which at least one amino acid in the amino acid sequence represented by SEQ ID NO:1 is deleted, substituted and/or added, and has granulocyte colony-stimulating factor activity.

4. The method according to claim 1, wherein the polypeptide is a polypeptide which consists of an amino acid sequence having a homology of 80% or more with the amino acid sequence represented by SEQ ID NO:1, and has granulocyte colony-stimulating factor activity.

5. The method according to claim 1, wherein the polypeptide is a chemically modified polypeptide having granulocyte colony-stimulating factor activity.

6. The method according to claim 5, wherein the modified polypeptide is a chemically modified polypeptide with polyalkylene glycol.

7. The method according to claim 1, wherein the bone marrow suppressed conditions are bone marrow suppressed conditions which are caused by administration of an antitumor agent having bone marrow suppressive activity.

8. The method according to claim 7, wherein the antitumor agent is at least one antitumor agent selected from the group consisting of an alkylating agent, a metabolic antagonist, an antitumor antibiotic, a plant pharmaceutical preparation and a platinum compound.

9. The method according to claim 7, wherein the administration of the antitumor agent is administration of an alkylating agent and a metabolic antagonist in combination.

10. The method according to claim 8, wherein the alkylating agent is cyclophosphamide.

11. The method according to claim 9, wherein the alkylating agent is cyclophosphamide.

12. The method according to claim 8, wherein the metabolic antagonist is 5-FU.

13. The method according to claim 9, wherein the metabolic antagonist is 5-FU.

14. The method according to claim 7, wherein the bone marrow suppressed conditions are bone marrow suppressed conditions in which count of leukocytes in peripheral blood is 90% or less of the count before the administration of the antitumor agent.

15. The method according to claim 7, wherein the bone marrow suppressed conditions are bone marrow suppressed conditions in which count of leukocytes in peripheral blood is 75% or less of the count before the administration of the antitumor agent.

16. The method according to claim 7, wherein the bone marrow suppressed conditions are bone marrow suppressed conditions in which count of leukocytes in peripheral blood is 60% or less of the count before the administration of the antitumor agent.

17. The method according to claim 1, wherein the heart diseases and vascular system diseases are at least one of diseases selected from the group consisting of myocardial infarction, angina pectoris, heart failure, cardiac myopathy and obstructive arteriosclerosis.
